(19) United States
(12) Patent Application Publication
(10) Pub. No.: US 2009/0281070 A1
(43) Pub. Date: Nov. 12, 2009

(54) Method for Preparing a Solid Pharmaceutical Composition with Sustained and Controlled Release by High Pressure Treatment

(76) Inventors: Vassilios Kaltsatos, Libourne (FR); Patrick Forget, Merignac (FR); Eliane Boivin, Bordeaux (FR)

Correspondence Address: JACOBSON HOLMAN PLLC, 400 SEVENTH STREET N.W., SUITE 600, WASHINGTON, DC, 20004, US

(21) Appl. No.: 11/794,542

(22) PCT Filed: Dec. 20, 2005
(86) PCT No.: PCT/FR05/03203

§ 371 Date: Jul. 2, 2007

(30) Foreign Application Priority Data
Dec. 30, 2004 (FR) 0414099

(51) Int. Cl. A61K 31/56

(52) U.S. Cl. 514/182

(57) ABSTRACT

The invention concerns a method for preparing a unit-dose of a solid pharmaceutical composition with prolonged and controlled release by high pressure treatment of a solid composition comprising at least one active principle and at least one polymer. The invention also concerns the resulting solid pharmaceutical compositions.

The present invention concerns a method for manufacturing a solid pharmaceutical composition with sustained and controlled release, by high pressure treatment of a composition comprising at least one active ingredient and at least one polymer.

The use of compositions having the property of releasing the active ingredients in a sustained and controlled manner is very frequent in the human and veterinary pharmaceutical fields. This makes it possible in a single dose to obtain a sustained effect of from several hours up to several months for certain implantable forms, especially ophthalmic inserts.

In order to obtain compositions having such properties, different types of formulations have been developed and various methods for manufacturing said compositions are employed.

Thus, in general, the active ingredients are mixed with polymers. By the application of a specific manufacturing method, said polymers form a matrix containing said active ingredients. The property of releasing the active ingredients in a sustained and controlled manner (or release kinetics) will therefore be closely correlated on the one hand to the nature of the polymers used to form the matrix, and on the other hand to the manufacturing method employed to obtain a matrix having the specific characteristics. In particular, it is preferable for the method used to enable the glass transition temperature of the polymers to be reached so that the polymer molecules melt with one another and arrange themselves spatially to form a homogeneous plastic matrix.

The “glass transition temperature” designates the temperature at which a polymer passes from a soft and flexible state to a hard (plastic) or even brittle state. Thus, in order that the active ingredient is well isolated in a polymer matrix, the manufacturing method must employ a heat treatment at a temperature above the glass transition temperature, in order to soften the polymer, produce the matrix by causing the polymer to assume a densified form, free of holes or pores, then the temperature must be reduced to below the glass transition temperature, in order to re-harden the polymer and render it resistant to external attacks, i.e. gas, water vapour, oxygen, water, etc.

Several preparation methods making it possible to impart to a composition properties of sustained and controlled release of said active ingredients are well known.

Thus, a method of compression in dry form is known, compression being able to be direct or double. The term “double compression” designates the technique known as double compression and that known as compacting and compression. This method has numerous drawbacks. In particular, large amounts of polymers are necessary to form the matrix around the active ingredient. In addition, since compression does not enable the glass transition temperature to be reached, it does not allow good “fusion” of the polymer molecules with one another. As a consequence, the matrix formed has a large number of inter-granular pores and of pores in the polymer film enveloping the active ingredient, which have a negative impact on the sustained and controlled release of the active ingredient.

Furthermore, a method of granulation by means of solvent, followed by compression, is known. This method, during granulation (owing to part or all of the polymer being made soluble in the solvent and to the use of plasticiser), makes it possible to lower the glass transition temperature and therefore form a film covering the active ingredient. But during compression, the forces involved do not permit enough fusion of the polymer to obtain the ideal matrix. This method does indeed allow retarded and sustained forms to be made, but these forms generally have dissolution times of less than 8 hrs.

Finally, a hot extrusion process is known. This type of process is most frequently used for the preparation of sustained release compositions. It consists in forcing through a die a composition comprising a plastic polymer previously softened by increasing the temperature, in order to form a continuous piece. During this process, high temperatures are applied, enabling the glass transition temperature to be reached, thereby making it possible to produce a sealed film around the active ingredients and to form a matrix devoid of inter-granular pores. This process makes it possible to obtain compositions permitting sustained and controlled release of the active ingredient over a significant time lapse. However, it has the drawback of being usable only for active ingredients that are stable at high temperature, since heat-sensitive active ingredients cannot be formulated in a sustained release form by this process. In fact, this technique requires the application of temperatures generally higher than 150° C.

High pressure treatments are used for methods of sterilisation of active ingredients or compositions. French Application FR 00 01059 describes a method for sterilising at least one active ingredient by high pressure treatment at between 3×10⁸ and 6×10⁸ Pa. Furthermore, French Application FR 02 05458 describes a method for sterilising without degradation a pharmaceutical composition in micronised or nanodispersed form comprising at least one active ingredient, by high pressure treatment at between 200 and 1000 MPa. High pressure treatments for sterilising foods, drugs, etc. are already well known to the skilled person. On the other hand, a high pressure treatment has never been used in a method for manufacturing a composition permitting sustained and controlled release of the active ingredient.

The present invention remedies the drawbacks inherent in the manufacturing methods discussed above.

The subject of the invention is a method for manufacturing solid pharmaceutical compositions with sustained and controlled release of at least one active ingredient, said method comprising the high pressure treatment of a solid composition comprising at least one active ingredient and at least one polymer.

The inventors have in fact demonstrated that the manufacture of solid pharmaceutical compositions by high pressure treatment makes it possible to obtain a homogenous composition permitting the sustained and controlled release of said at least one active ingredient.

### Definitions

By “composition” there is meant any composition in solid form. Thus, the compositions in solid form comprise in particular, on the one hand, unit-doses such as tablets, implants, inserts, lozenges, suppositories, etc. and on the other hand pulverulent solids such as powders. By “insert” there is meant in particular a solid or semi-solid sterile preparation, of suitable size and shape, intended to be inserted into the conjunctival sac for the purpose of action on the eye (see for example Inserenda Ophtalmica, Monograph 830, European Pharmacopoeia, 2004, 4th edition).

“Sustained release” is to be understood as making an active ingredient or ingredients available to the organism, in a constant or programmed manner over periods of from 8 to 12 hours to several months (for implants). Within the framework of the present application, the expression “controlled release” characterizes the release of the active ingredient according to a linear kinetic (zero order kinetic), that is to say, the active ingredient is released at a constant speed in the medium over time.

By “high pressure” there is meant a treatment carried out at pressures generally above 100 MPa (10⁸ Pa).

By “heat-sensitive” there is meant an active ingredient of which the biological activity is diminished, partially or totally, following a high temperature treatment applied for several hours, for example a treatment at 100° C. for 15 hours.

### Method for Manufacturing Pharmaceutical Compositions With Sustained and Controlled Release

The present invention therefore concerns a method for manufacturing a solid pharmaceutical composition with sustained and controlled release by high pressure treatment of a composition comprising at least one active ingredient and at least one polymer.

More precisely, said method comprises the step which consists in subjecting a solid composition comprising at least one active ingredient and at least one polymer to a pressure above 10⁸ Pa and to a heat treatment for a specific duration. The heat treatment is generally carried out at a temperature above +10° C. The duration of said step is generally one minute or more.

Preferably, the solid composition is subjected to a pressure of between 10⁸ and 10⁹ Pa, more preferably between 10⁸ and 6×10⁸ Pa, and even more preferably between 2.5×10⁸ and 5×10⁸ Pa.

Preferably, the temperature of the heat treatment is between +10° C. and +150° C., more preferably between +20° C. and +120° C., even more preferably between +45° C. and +100° C., and yet more preferably between +60° C. and +90° C.

Preferably, the duration of said step is between one minute and one hour, and more preferably between 1 and 30 minute(s).

Thus, in a particularly preferred embodiment, said method comprises the step which consists in subjecting a solid composition comprising at least one active ingredient and at least one polymer to a pressure of between 10⁸ and 6×10⁸ Pa, and to a temperature of between +20° C. and +120° C. for a duration of between one minute and 30 minutes.

More specifically, in order to obtain a matrix having satisfactory properties, it is desirable for the glass transition temperature of the polymer or polymers to be reached during the course of the manufacturing method. In fact, when the glass transition temperature is reached, the polymers “melt” and become malleable, with a plastic behaviour, and then form a homogeneous matrix devoid (or almost devoid) of inter-granular pores. This temperature depends on the nature of the polymer or polymers used. The glass transition temperatures of the polymers are given by the documents of the polymer supplier. In fact, these temperatures depend very much on the nature of the polymer used (molecular weight, crosslinking, groups and their location, sequences), and on the method employed for obtaining them (solvents used, manufacturing method). In cases where this value is not known, it may be determined by differential thermal analysis (DTA) or by differential scanning calorimetry (DSC), which are well known to the skilled person (see for example “Pharmaceuticals Thermal Analysis—Techniques and applications”, pp. 9 to 14-pp. 29 to 31, Ford J. and Timmins P., 2nd edition, Taylor & Francis, ISBN 0748407618). It should be noted in particular that the glass transition temperature of a polymer depends on the proportion of crystalline form and amorphous form in said polymer.

The composition subjected to the method according to the invention may contain an active ingredient of any kind. For example, the composition may comprise at least one active ingredient which may belong, in particular, to the class of antibiotics, to the class of non-steroidal and corticosteroidal anti-inflammatories, the class of hormones or hormonal analogues, the class of anti-cancer agents, the class of antiviral agents, the class of drugs for the central nervous system (anti-migraine, sedative, hypnotic, anti-Parkinson, anti-epileptic, antidepressant agents). Preferably, the composition comprises at least one active ingredient belonging especially to the class of antibiotics. Finally, particularly preferably, the composition comprises fusidic acid.

The composition further comprises at least one polymer which is a biocompatible polymer. Depending on the nature of the active ingredient and/or on the method of administration, the biocompatible polymers may be water-soluble or insoluble in water. Different types of polymers are particularly adapted to the implementation of the method according to the invention, that is to say, polymers belonging especially to the acrylate derivatives, polycarbophilic derivatives, cellulose derivatives, vinyl derivatives (vinyl acetate, vinyl alcohol, vinyl acetate phthalate, which may or may not be associated with ethylene molecules) or vinyl pyrrolidine, to gums (lac, adragante, xanthan, guar, gum arabic, tragacanth, scleroglucans, carrageenans), to derivatives of polysaccharides, to polyoxyethylene derivatives, to derivatives of polyethylene-polypropylene glycol (poloxamers), dextran derivatives, to gelatin derivatives, derivatives of sugars and phenols, derivatives of alginic acids, silicone derivatives, derivatives of lactic and glycolic acids, to polyanhydride derivatives, polybutadiene derivatives, glutamic acid derivatives, polyorthoester derivatives and/or to derivatives of ion exchange resins. Preferably, the polymers belong to the family of cellulose derivatives. Particularly preferably, the polymers are selected from hydroxyethylcellulose, hydroxypropylmethylcellulose or ethylcellulose.

The respective proportions of active ingredient(s) and polymer(s) depend on the nature of the active ingredients and polymers used. Thus, in the presence of an active ingredient having a very low solubility in an aqueous medium, it is not necessary to introduce a large quantity of polymers into the composition. In fact, the speed of dissolution of a solid active ingredient in a medium is proportional to its solubility in said medium. As a consequence, an active ingredient poorly soluble in a medium will have a low speed of dissolution in that medium, the release of the active ingredient then being sustained over time. The determination of these respective proportions is within the scope of the skilled person.

Optionally, the composition further comprises various pharmaceutically acceptable excipients. By pharmaceutically acceptable “excipient” there is meant any compound able to facilitate the shaping of the composition and not modifying the nature of the biological activity of the active ingredient. A pharmaceutically acceptable excipient may be a solvent, plasticiser, lubricant, dispersion medium, absorption retardants, flow agent, etc. Preferably, the composition further comprises plasticisers, lubricants and/or flow agents.

Thus, the composition further optionally comprises at least one plasticiser, in particular when the polymer is difficultly compressible. Different types of plasticisers are particularly adapted to the implementation of the method according to the invention, i.e. polyethylene glycol derivatives, vegetable or mineral oils, phthalic or sebacic derivatives, triacetin, triethylcitrate and fatty substances. Preferably, the plasticisers are vegetable oils. Particularly preferably, the plasticiser is castor oil.

Moreover, the composition further optionally comprises at least one lubricant. Different types of lubricant are particularly adapted to the implementation of the method according to the invention, i.e. magnesium stearate, stearic acid, leucine, glycine, sodium lauryl sulphate, sodium benzoate, potassium benzoate, sodium stearyl fumarate, glycerol benate, hydrogenated vegetable oil, polyethylene glycol, silicone, talc, zinc stearate, glycerine monostearate and glycerol palmitostearate. Preferably, the lubricant is a hydrogenated vegetable oil.

Preferably, the solid composition comprises, by weight based on the total weight of the composition:
10 to 40% of active ingredient, in particular fusidic acid;
50 to 90% of polymer(s) and optionally one or more pharmaceutically acceptable excipients.

More preferably, the solid composition comprises, by weight based on the total weight of the composition:
20 to 30% of active ingredient, in particular fusidic acid;
60 to 80% of polymer(s) and optionally one or more pharmaceutically acceptable excipients.

According to a preferred embodiment, said solid composition subjected to the method according to the invention comprises 25% of fusidic acid, 1% of hydroxyethylcellulose, 65.5% of hydroxypropylmethylcellulose, 5% of ethylcellulose, 3% of hydrogenated vegetable oil and 0.5% of castor oil, the percentages being expressed by weight based on the total weight of the composition. Particularly preferably, said solid composition is an ophthalmic insert. Said insert may then be inserted directly into the conjunctival sac. Nevertheless, prior to any use on an animal of the high pressure treated solid composition, this composition may optionally be sterilised (by radiation or otherwise) and/or may be packaged (sachet, blister pack).

The composition comprising at least one active ingredient, at least one polymer and optionally one or more excipients, which is subjected to the method according to the invention, is in solid form. Advantageously, prior to the high pressure treatment, the different compounds (at least one active ingredient, at least one polymer and optionally the excipients) forming the composition are mixed to form a pulverulent solid form. The pulverulent solid form is optionally granulated by the granulation techniques well known to the skilled person, in order to form a granulated solid form. Finally, the pulverulent solid forms or the granulated solid forms are optionally compressed, moulded or cast by techniques well known to the skilled person in order to obtain a solid unit-dose.

Preferably, the different compounds forming the composition are mixed and undergo granulation before being transformed into a solid unit-dose.

The solid form (pulverulent, granulated or unit-dose) is then high pressure treated in order to obtain a composition permitting sustained and controlled release of the active ingredient.

The high pressure treatment is applied in such a way as to induce a homothetic reduction of the dimensions of the composition. By “homothetics” (or scaling down) there is meant the modification of an object by reduction or enlargement thereof proportionally in all spatial directions. The homothetic reduction does not therefore involve any change of shape. Such a reduction without modification of shape is obtained by the application of an isostatic pressure, that is to say, during the course of the treatment the pressure applied is identical at every point of the surface area of the solid form.

The high pressure treatment may be implemented by using an isostatic press apparatus, preferably of the hot type, so as to be able also to apply a heat treatment. For example, a hot isostatic press on an industrial scale marketed by NovaSwiss® is suitable for implementing the invention.

The use of high pressure is generally combined with specific temperatures. The choice of temperature is directly correlated to the nature of the polymer or polymers used. The application of an adequate temperature permits the modification of the polymers used and, in particular, the modification of their physical characteristics. In fact, the application of a temperature above the glass transition temperature of the polymer permits the formation of a homogeneous and sealed matrix by fusion of the polymers and condensation of these latter around the active ingredient.

Moreover, the inventors have observed that the application of high pressures makes it possible to reduce the glass transition temperature of the polymers compared with the hot extrusion process of the prior art. This reduction is important, since it makes it possible to work with active ingredients that are sensitive to heat and therefore to apply the method according to the invention to the manufacture of compositions comprising at least one heat-sensitive active ingredient.

The combined application of the high pressures and of an adequate temperature results in a great reduction in the porosity of the composition obtained. In particular, a decrease in the number of intra-granular and inter-granular pores, a reduction in the surface area/volume ratio and an increase in the density of the composition are observed. These modifications directly influence the profile over time of the release of the active ingredient, as well as a great reduction in the penetration of water and gases into the composition.

Thus, the method according to the invention, after having incorporated the active ingredient by mixing followed optionally by granulation and/or compacting, then optionally compression, in a matrix formed substantially of polymers, will, by the effect of a high pressure treatment, effect the closure of a large number of intra-granular pores and ensure the continuity and tightness of the polymeric matrix. The continuity and tightness impart to the polymeric matrix qualities permitting the regulation and prolongation of the dissolution of the active ingredient. The concept obtained permits dissolutions at least as sustained as those of the products obtained by fusion-extrusion.

It has the advantage, during the process, of requiring much lower temperatures than those of the fusion-extrusion technique.

In addition, during granulation, it makes it possible to have granulation phases with different polymers and solvents, thus offering a very wide palette of possible properties depending on the types and qualities of the polymers used.

### Solid Pharmaceutical Compositions With Sustained and Controlled Release

Another subject of the invention concerns the pharmaceutical compositions with sustained and controlled release that are obtainable by the method according to the invention.

According to a particular embodiment, said solid pharmaceutical composition comprises at least one heat-sensitive active ingredient and at least one polymer.

Said pharmaceutical composition permits the sustained and controlled release of said at least one active ingredient for a duration of at least 8 hours, preferably of at least 12 hours or, more preferably, for one, two or several months.

According to a preferred embodiment, the pharmaceutical composition according to the invention comprises fusidic acid as the active ingredient. Advantageously, the pharmaceutical composition comprises at least one polymer which is selected from the group consisting of hydroxymethylcellulose, hydroxymethylpropylcellulose and ethylcellulose.

Preferably, the pharmaceutical composition comprises 25% of fusidic acid, 1% of hydroxyethylcellulose, 65.5% of hydroxymethylpropylcellulose, 5% of ethylcellulose, 3% of hydrogenated vegetable oil and 0.5% of castor oil.

The present invention will be more easily understood from the figure and the example below which are provided by way of non-limiting example of the invention.

### FIGURE

FIG. 1 shows the analysis of the release profile, by dissolution in a phosphate buffer medium, of the active ingredient contained in a composition not high pressure treated and in a composition according to the invention.

### EXAMPLES

### Example 1

### Comparison of the Technical Characteristics of High Pressure Treated Solid Forms and of Untreated Solid Forms

The solid forms are obtained by consecutive granulations of 25% of fusidic acid with 65.5% of hydroxymethylpropylcellulose, 5% of ethylcellulose, 1% of hydroxyethylcellulose, 3% of hydrogenated vegetable oil and 0.5% of castor oil, by weight based on the total weight of the mixture, then by compression in order to obtain a solid unit-dose of fusidic acid of cylindrical shape, the dimensions of which are indicated in the table below. The steps of granulation and compression are carried out with conventional apparatuses.

The solid forms are treated according to the present invention, by the use of the apparatus “Pilote HP tout Inox 7000 bars” (reference 7-1000-097, marketed by Novaswiss®). In order so to do, the solid forms are placed in sealed plastic envelopes and are immersed in water contained within the isostatic enclosure. The solid forms are then treated by the application of an isostatic pressure of 5.10⁸Pa at a temperature of 80° C. for 10 minutes. More precisely, said method carried out comprises a first phase of regular pressure increase of 5 minutes, followed by a second, plateau phase by the application of an isostatic pressure of 5×10⁸ Pa at a temperature of 80° C. for 10 minutes and a decompression phase of 50 seconds.

Only the 10 minute stationary phase has an influence on the improvement of the profile of sustained and controlled release.

The characteristics of the treated solid forms that are obtained are compared to those of the untreated solid forms. In particular, the mass, thickness, diameter, surface area, volume and surface area/volume ratio of the treated and untreated solid forms were calculated on the basis of methods well known to the skilled person.

The results obtained are detailed in Table 1 below.

| | Untreated solid | Treated solid |
|---|---|---|
| Characteristics | forms | forms |
| Mass | 25.17 mg | 25.17 mg |
| | (cv = 3.33%) | (cv = 3.33%) |
| Thickness | 5.81 mm (cv = 0.49%) | 5.50 mm (cv = 0.02%) |
| Diameter | 2.4 mm | 2.19 mm |
| Surface area | 52.79 mm² | 45.38 mm² |
| Volume | 26.28 mm³ | 20.72 mm³ |
| Surface area/ | 2.01 | 2.19 |
| volume ratio | | |

“cv”: coefficient of variation [standard deviation/mean × 100]

As expected, the dimensions of the treated solid forms are reduced by the application of the high pressures.

The measurement of the surface area and volume shows that the ratio of these two parameters is higher for the treated solid forms than for the untreated solid forms. As a consequence, the increase in the surface area/volume ratio demonstrates that the volume decreases more quickly than the surface area. This increase characterises the significant contraction of the polymer matrix on the active ingredient. Since the mass of the treated composition is unchanged, the increase in the surface area/volume ratio therefore involves a reduction in the contact between the external medium and the treated solid form. Since this contact surface area is reduced, this retards and prolongs the release of the active ingredient. The accompanying effects of contraction of the polymer on the active ingredient and of a reduction in the surface area of contact with the external medium lead to an increase in the dissolution time. This is necessarily due to the disappearance of the inter-granular pores, the temperature applied during the treatment having modified the polymer, said polymer having then perfectly isolated the active ingredient under a sealed film of polymer.

### Example 2

### Comparison of the Dissolution Property of the Untreated Solid Forms and of the High Pressure Treated Solid Forms

Said treated and untreated solid forms are dissolved in a litre of phosphate buffer medium of pH=7.5 and at 37° C. (6.8 g/l of potassium dihydrogenphosphate adjusted to pH 7.5 with 35% soda). Said medium is agitated by means of blades having a rotation sped of 100 rpm.

The measurment of the active ingredient was carried out by HPLC. Measurement is performed on a C18 Kromasil® inverted phase column with a mobile phase constituted by a mixture of 9 volumes of methanol, 9 volumes of water, 16.4 volumes of phosphoric acid at 10 g/l and 65.6 volumes of acetonitrile. Detection is carried out by means of an ultraviolet spectrophotometer set at 235 nm (the retention time of fusidic acid is 5.98 min.). It should be noted that the “Sink” conditions were verified for fusidic acid. “Sink conditions” designates the experimental conditions making it possible to effect the dissolution of a pharmaceutical form without the dissolution being checked by the saturation of the medium and the active ingredient dissolution. Thus, under these conditions, it should be possible to render soluble, in the volume of medium defined, three times the amount of active ingredient normally present in the pharmaceutical form.

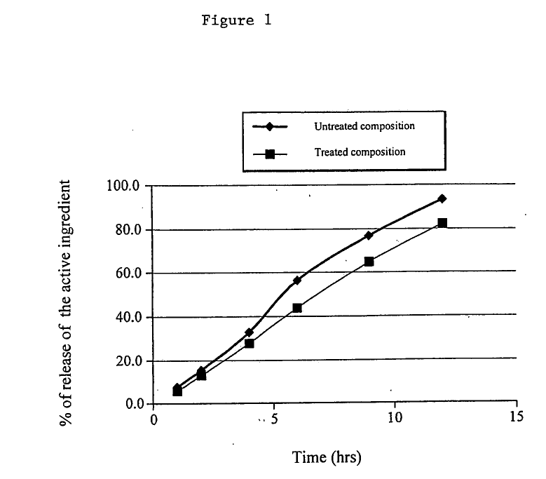

The results obtained are illustrated in FIG. 1 appended to the present Application.

It is observed that the release of the active ingredient contained in the treated solid forms is sustained by at least 2 hours compared with the untreated solid forms. Similarly, it is observed that this sustained release is accompanied by a transformation of the dissolution into a profile of the order 0. In other words, this means that the quantity of active ingredient released from the treated solid form is linear over time (controlled liberation) as opposed to the untreated solid form, for which the release profile is irregular over time. Similarly, when the rate of release of the active ingredient is expressed over time (amount of active ingredient released over time), this rate is constant in the case of a form with release of the order 0 and a horizontal straight line is therefore obtained.

1-20. (canceled)

21. A method for manufacturing a solid pharmaceutical composition with sustained and controlled release comprising the step which consists in subjecting a solid composition comprising at least one active ingredient and at least one polymer to a pressure above 10⁸ Pa and to a heat treatment at a temperature above +10° C. for a duration of at least one minute.

22. A manufacturing method according to claim 21, wherein said solid composition comprising at least one active ingredient and at least one polymer is subjected to a pressure of between 10⁸ and 10⁹ Pa.

23. A manufacturing method according to claim 21, wherein the heat treatment is carried out at a temperature of between +10° C. and +150° C.

24. A manufacturing method according to claim 21, wherein the duration of said step is between one minute and one hour.

25. A manufacturing method according to claim 21, wherein said solid composition comprising at least one active ingredient and at least one polymer is subjected to a pressure of between 10⁸ and 6×10⁸ Pa, at a temperature of between +20° C. and +120° C. for a duration of between one minute and 30 minutes.

26. A manufacturing method according to claim 21, wherein said composition comprising at least one active ingredient and at least one polymer is in a solid form selected from the group consisting of solid unit-doses such as tablets, implants, inserts, lozenges, suppositories and pulverulent solid forms such as powders.

27. A method according to claim 21, wherein said at least one active ingredient is selected from the group consisting of the active ingredients belonging to the class of antibiotics, the class of non-steroidal and corticosteroidal anti-inflammatories, the class of hormones or hormonal analogues, the class of anti-cancer agents, the class of anti-viral agents, and to the class of drugs for the central nervous system.

28. A method according to claim 27, wherein said at least one active ingredient is selected from the active ingredients belonging to the class of antibiotics.

29. A method according to claim 28, wherein said at least one active ingredient is fusidic acid.

30. A method according to claim 21, wherein said at least one polymer is selected from the group consisting of acrylate derivatives, polycarbophilic derivatives, cellulose derivatives, vinyl derivatives or vinyl pyrrolidone, gums, derivatives of polysaccharides, polyoxyethylene derivatives, derivatives of polyethylene-polypropylene glycol, dextran derivatives, gelatine derivatives, derivatives of sugars and phenols, derivatives of alginic acid, silicone derivatives, derivatives of lactic and glycolic acids, polyanhydride derivatives, polybutadiene derivatives, glutamic acid derivatives, polyorthoester derivatives and derivatives of ion exchange resins.

31. A method according to claim 30, wherein said at least one polymer belongs to the family of cellulose derivatives.

32. A method according to claim 31, wherein said solid composition comprising at least one active ingredient and at least one polymer comprises hydroxyethylcellulose, hydroxymethylpropylcellulose and/or ethylcellulose.

33. A method according to claim 21, wherein said solid composition comprising at least one active ingredient and at least one polymer further comprises at least one pharmaceutically acceptable excipient.

34. A method according to claim 33, wherein the excipient is selected from the group consisting of plasticisers, lubricants and flow agents.

35. A method according to claim 34, wherein the plasticiser is selected from the group consisting of polyethylene glycol derivatives, vegetable or mineral oils, phthalic or sebacic derivatives, triacetin, triethylcitrate and fatty substances.

36. A method according to claim 34, wherein the plasticiser is a vegetable oil.

37. A method according to claim 34, wherein the lubricant is selected from the group consisting of magnesium stearate, stearic acid, leucine, glycine, sodium lauryl sulphate, sodium benzoate, potassium benzoate, sodium stearyl fumarate, glycerol benate, hydrogenated vegetable oil, polyethylene glycol, silicone, talc, zinc stearate, glycerine monostearate and glycerol palmitostearate.

38. A method according to claim 33, wherein said solid composition comprising at least one active ingredient and at least one polymer comprises 25% of fusidic acid, 1% of hydroxyethylcellulose, 65.5% of hydroxymethylpropylcellulose, 5% of ethylcellulose, 3% of hydrogenated vegetable oil and 0.5% of castor oil.

39. A solid pharmaceutical composition with sustained and controlled release obtainable by the method according to claim 21.

40. A pharmaceutical composition according to claim 39, wherein it is an ophthalmic insert.
